(19) United States
(12) Patent Application Publication
(10) Pub. No.: US 2010/0228496 A1
(43) Pub. Date: Sep. 9, 2010

(54) Methods for the Determination of a Copy Number of a Genomic Sequence in a Biological Sample

(75) Inventors: Harrison M. Leong, San Francisco, CA (US); Catalin Barbacioru, Fremont, CA (US); Gordon A. Janaway, Hayward, CA (US)

Correspondence Address: LIFE TECHNOLOGIES CORPORATION;C/O INTELLEVATE, P.O. BOX 52050, MINNEAPOLIS, MN, 55402, US

(73) Assignee: LIFE TECHNOLOGIES CORPORATION, Carlsbad, CA (US)

(21) Appl. No.: 12/720,595

(22) Filed: Mar. 9, 2010

Related U.S. Application Data
(60) Provisional application No. 61/158,718, filed on Mar. 9, 2009.

(51) Int. Cl. G06F 17/18; C12Q 1/68; G06F 19/00

(52) U.S. Cl. 702/19; 435/6; 702/181

(57) ABSTRACT

Methods for the determination of a copy number of a target genomic sequence; either a target gene or genomic sequence of interest, in a biological sample are described. Various methods utilize a model drawn from a probability density function (PDF) for the assignment of a copy number of a target genomic sequence in a biological sample. Additionally, the methods provide for the determination of a confidence value for a copy number assigned to a sample based on attributes of the sample data. Accordingly, the various methods for the determination of a copy number provide the end user with significant information for the evaluation of a copy number of a target genomic sequence; either a gene or genomic sequence of interest.

### FIELD

The field of disclosure relates to methods for determining the copy number of a biological sample with a defined confidence.

### BACKGROUND

The polymerase chain reaction (PCR) represents an extensive family of chemistries that have produced numerous types of assays of impact in biological analysis. Accordingly, concomitant to the innovation of assays for this family of chemistries has been the innovation of computational methods matched to the objectives of the various PCR-based assays.

For example, one type of computational method suited to various types of quantitative PCR (qPCR) assays is often referred to as the comparative threshold cycle (Cₜ) method. As one of ordinary skill in the art is apprised, the cycle threshold, Cₜ, indicates the cycle number at which an amplified target genomic sequence; either a gene or genomic sequence of interest, reaches a fixed threshold. A relative concentration of a target genomic sequence; either a gene or genomic sequence of interest, may be determined using Cₜ, determinations for the target genomic sequence, a reference genomic sequence; of which for many qPCR assays may be either an endogenous or exogenous reference genomic sequence, and additionally, a calibrator sequence. After normalizing the Cₜ, data for the target gene sequence and the calibrator gene sequence to the reference gene sequence samples, under the assumption that the efficiencies of the reactions are equal and essentially 100%, one of ordinary skill in the art would recognize the calculation for the comparative Cₜ method as:

X_{N,t}/X_{N,c}=2^{-ΔΔCt};where
X_{N,t}/X_{N,c}=is the relative concentration of the target in comparison to the calibrator; and
ΔΔCₜ=is the normalized difference in threshold cycles for the target and the calibrator

In practice, the efficiency of the PCR process may not be exactly 100%, as the concentration of genetic material may not double at every cycle. Factors that may affect the efficiency of an amplification reaction may include, for example, reaction conditions such as the difference in the detection limit for the dye used for a target genomic sequence versus the dye for the reference, or in inherent differences in the sequence context of the target genomic sequence and a reference genomic sequence. However, as assays are optimized to ensure the highest efficiencies, any deviations from the assumption of 100% efficiency are generally small. In addition to possible deviations from ideality, there are variations of replicate samples of the same sequence, due to variations contributions in an assay system from both the chemistry and instrumentation.

Accordingly, various methods for the determination of a gene copy use statistical models to assign a copy number to a sample in a population of samples, and determine a confidence value to the assignment. Such methods take into account various assay deviations and variations. Unlike the comparative Cₜ method, or ΔΔCₜ method, as it is often referred, various methods for the determination of a gene copy number utilize the information in ΔCₜ determinations of samples, and therefore do not require the use of a calibration sample data.

### BRIEF DESCRIPTION OF THE DRAWINGS

FIG. 1 is a flow chart that depicts various embodiments of methods for the determination of the copy number for a biological sample.

FIG. 2 depicts various embodiments of an apparatus useful in the generation of data for a biological sample for which a copy number determination is desired.

FIG. 2 depicts

FIG. 4A-FIG. 4C depict a step of assigning copy number for various embodiments of methods for the determination of gene copy number in a biological sample.

FIG. 5 is an exemplary probability sample frequency population after various embodiments of methods for the determination of gene copy number, which was estimated using a probability density function model based on a normal distribution.

FIG. 6A and FIG. 6B are graphs that depict the confidence values of a copy number call for various embodiments of methods for the determination of the copy number related to

FIG. 7A and FIG. 7B are graphs that depict the confidence values of a copy number call for various embodiments of methods for the determination of the copy number related to probability density graphs for a set of copy number calls for two sample sets having the same probability densities but different sample variances.

FIG. 8A is a table depicting the confidence values at 95% probability of a copy number call for various embodiments of methods for the determination of the copy number as a function of copy number and sample standard deviation. FIG. 8B depicts the distribution of a plurality of sample determinations having targeted sample variances, as shown in FIG. 8A.

FIG. 9 is a chart depicting the assignment of copy number for a plurality of samples using various embodiments of methods for the determination of the copy number in comparison to an established method for assignment of copy number.

### DETAILED DESCRIPTION

What is disclosed herein are various embodiments of methods for the determination of a copy number of a target genomic sequence; either a target gene or genomic sequence of interest, in a biological sample. In various embodiments, a model drawn from a probability density function (PDF) may be used as the basis for the assignment of a copy number of a target genomic sequence in a biological sample. According to various embodiments, the parameter space defining the selected PDF model is searched until values for the parameters that define the PDF model are optimized to a measure of fit between the observed sample data and the PDF model. Additionally, various embodiments provide for the determination of a confidence value for a copy number assigned to a sample based on attributes of the sample data. Accordingly, a confidence value so determined may provide for an independent evaluation of the assigned copy number generated using a PDF model. In that regard, the various embodiments of methods for the determination of a copy number disclosed herein provide the end user with significant information for the evaluation of a copy number of a target genomic sequence; either a gene or genomic sequence of interest.

The type of assay that is used to provide the data for various embodiments of methods for the determination of a copy number is known to one of ordinary skill in the art as the real-time quantitative polymerase chain reaction (real-time qPCR). Though subsequent examples provided may utilize an assay format known as TaqMan®, various methods for the determination of a copy number may be used with any assay that provides quantitative data. For example, but not limited by, one of ordinary skill in the art would recognize Molecular Beacons, Amplifluor® Primers, Scorpion™ Primers, Plexor™ Primers, and BHQplus™ Probes as providing assay formats for qPCR. In that regard, any assay format that is a sequence-specific qPCR assay format may be used to provide data for various embodiments of methods for the determination of a copy number of a target genomic sequence in a biological sample.

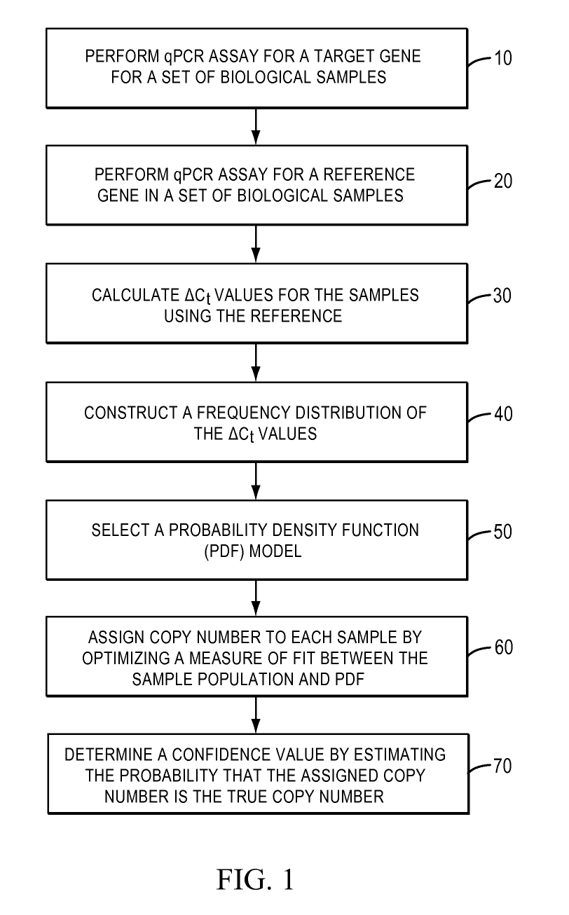

According to steps 10 and 20 of FIG. 1, a qPCR assay for a target genomic sequence may be run on a plurality of samples, where each sample may be run in a plurality of replicates. For the determination of copy number using qPCR, run simultaneously with the samples, an endogenous reference genomic sequence; either an endogenous reference gene or endogenous reference genomic sequence, having a known number of copies is also assayed. It is desirable for the endogenous reference genomic sequence to have little observed variation in copy number for in the population from which the samples of interest are drawn. For example, the RNase P H1 RNA gene is known to exist in two copies in a human diploid gene.

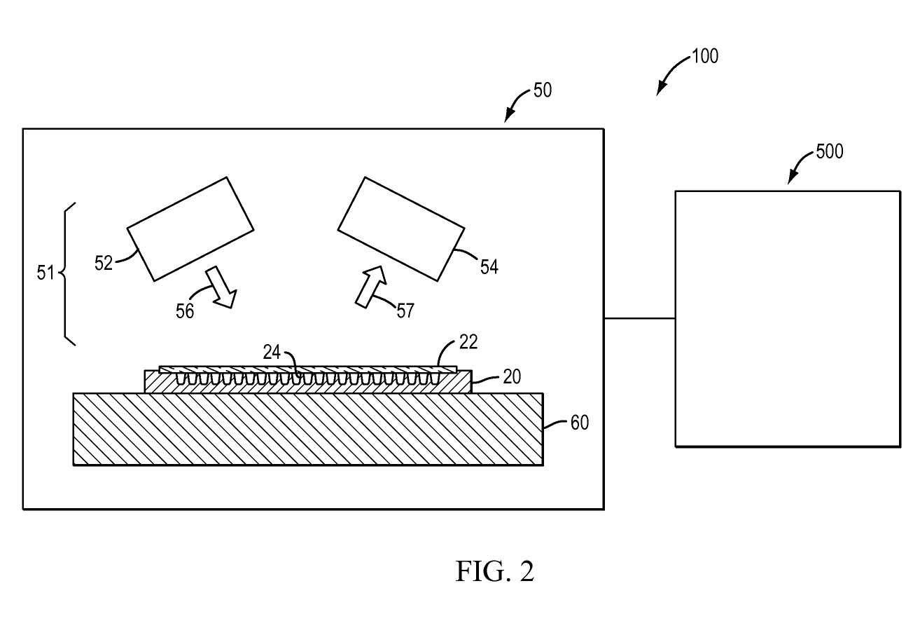

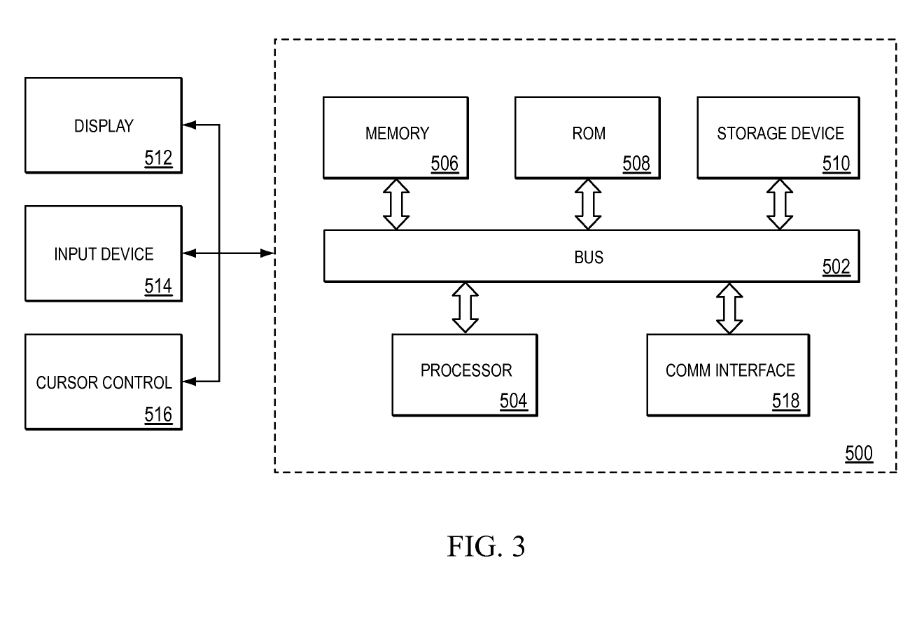

To perform 10 and 20 of FIG. 1, various embodiments of an assay system as depicted in FIG. 2 may be used. According to various embodiments of system 100, as shown in FIG. 2, a sample with a target genomic sequence can be loaded into a sample support device 20 of thermal cycler apparatus 50. Various embodiments of a sample support device may have a plurality of sample regions. In various embodiments, sample support device 20 may be a glass or plastic slide with a plurality of sample regions 24, which may be isolated from the ambient by cover 22. Some examples of a sample support device may include, but are not limited by, a multi-well plate, such as a standard microtiter 96-well, a 384-well plate, or a microcard. The sample regions in various embodiments of a sample support device may include depressions, indentations, ridges, and combinations thereof, patterned in regular or irregular arrays on the surface of the substrate. As depicted in FIG. 2, a sample support device may be placed in a thermal cycler apparatus 50. In various embodiments of a thermal cycler apparatus, there may be a heat block, 60, and a detection system 51. The detection system 51 may have an illumination source 52 that emits electromagnetic energy 56, and a detector 54, for receiving electromagnetic energy 57 from samples in sample support device 20. Computer system 500 can control the function of the thermal cycler apparatus. Additionally, computer system 500 may provide data processing and report preparation functions. All such instrument control functions may be dedicated locally to the thermal cycler apparatus 50, or computer system 500 may provide remote control of part or all of the control, analysis, and reporting functions, as will be discussed in more detail subsequently.

FIG. 3 is a block diagram that illustrates a computer system 500, according to various embodiments, upon which embodiments of methods for the analysis of PBA data may be implemented. Computer system 500 includes a bus 502 or other communication mechanism for communicating information, and a processor 504 coupled with bus 502 for processing information. Computer system 500 also includes a memory 506, which can be a random access memory (RAM) or other dynamic storage device, coupled to bus 502 for determining base calls, and instructions to be executed by processor 504. Memory 506 also may be used for storing temporary variables or other intermediate information during execution of instructions to be executed by processor 504. Computer system 500 further includes a read only memory (ROM) 508 or other static storage device coupled to bus 502 for storing static information and instructions for processor 504. A storage device 510, such as a magnetic disk or optical disk, is provided and coupled to bus 502 for storing information and instructions.

Computer system 500 may be coupled via bus 502 to a display 512, such as, but not limited by, a cathode ray tube (CRT), liquid crystal display (LCD), or light-emitting diode (LED) for displaying information to a computer user. However, one of ordinary skill in the art may readily recognize that there are various ways of outputting data to an end user in a variety of forms, for example, but not limited by, having a report sent to a printer. An input device 514, including alphanumeric and other keys, is coupled to bus 502 for communicating information and command selections to processor 504. Another type of user input device is cursor control 516, such as a mouse, a trackball or cursor direction keys for communicating direction information and command selections to processor 504 and for controlling cursor movement on display 512. This input device typically has two degrees of freedom in two axes, a first axis (e.g., x) and a second axis (e.g., y), that allows the device to specify positions in a plane. A computer system 500 provides base calls and provides a level of confidence for the various calls. Consistent with certain implementations of the invention, base calls and confidence values are provided by computer system 500 in response to processor 504 executing one or more sequences of one or more instructions contained in memory 506. Such instructions may be read into memory 506 from another computer-readable medium, such as storage device 510. Execution of the sequences of instructions contained in memory 506 causes processor 504 to perform the process states described herein. Alternatively hard-wired circuitry may be used in place of or in combination with software instructions to implement the invention. Thus implementations of the invention are not limited to any specific combination of hardware circuitry and software.

The term “computer-readable medium” as used herein refers to any media that participates in providing instructions to processor 504 for execution. Such a medium may take many forms, including but not limited to, non-volatile media, volatile media, and transmission media. Non-volatile media includes, for example, optical or magnetic disks, such as storage device 510. Volatile media includes dynamic memory, such as memory 506. Transmission media includes coaxial cables, copper wire, and fiber optics, including the wires that comprise bus 502. Transmission media can also take the form of acoustic or light waves, such as those generated during radio-wave and infra-red data communications.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, any other memory chip or cartridge, a carrier wave as described hereinafter, or any other medium from which a computer can read.

Various forms of computer readable media may be involved in carrying one or more sequences of one or more instructions to processor 504 for execution. For example, the instructions may initially be carried on magnetic disk of a remote computer. The remote computer can load the instructions into its dynamic memory and send the instructions over a telephone line using a modem. A modem local to computer system 500 can receive the data on the telephone line and use an infra-red transmitter to convert the data to an infra-red signal. An infra-red detector coupled to bus 502 can receive the data carried in the infra-red signal and place the data on bus 502. Bus 502 carries the data to memory 506, from which processor 504 retrieves and executes the instructions. The instructions received by memory 506 may optionally be stored on storage device 510 either before or after execution by processor 504.

As depicted in step 30 of FIG. 1, the Cₜ values between the threshold cycles of the target and endogenous reference sequences are used to calculate ΔCₜ each sample analyzed. According to various embodiments, the ΔCₜ values are used as the basis for fit in the

By way of providing an overview of the calculations for ΔCₜ and ΔΔCₜ for a copy number assay, the calculation of ΔCₜ values from a data set is based on the equation for the progress of reaction for a PCR assay. It is well know that for a PCR reactions the equation describing the exponential amplification of PCR is given by:

Xₙ=Xₒ[(1+E_{X})ⁿ]where: (EQ. 1)
Xₙ=the number of target molecules at cycle n
Xₒ=the initial number of target molecules
E_{X}=the efficiency of the target amplification
n=the number of cycles
from that relationship, the concentration of a genomic sequence at the threshold is:

X_{Ct,x}=Xₒ[(1+E_{X})^{Ct,x}=K_{X} where: (EQ. 2)
X_{Ct,x}=the number or target molecules at Cₜ
Xₒ=the initial number of target molecules
E_{X}=the efficiency of the target amplification
C_{t,x}=the number of cycles at Cₜ
K_{X}=a constant

From this it is evident that for a target genomic sequence; either a gene or genomic sequence of interest, the concentration of target formed in the reaction at Cₜ is a constant K, and therefore characteristic of the reaction. Generally, K may vary for various target genomic sequences, due to a number of reaction variables, such as, for example the reporter dye used in a probe, the efficiency of the probe cleavage, and the setting of the detection threshold. Additionally, as previously described, is generally held that the assumption that the efficiencies of reactions are optimized and essentially the same. Under such conditions and assumptions, it can be shown through the algebraic manipulation of EQ. 2, that normalizing a target genomic sequence of interest of to an endogenous reference reaction at Cₜ yields the following relationship:

X_{N}=K[(1+E)^{-ΔC}]where: (EQ. 3)
X_{N}=is the normalized amount of the target
ΔCₜ=is the difference in threshold cycles for the target and endogenous reference genomic sequence

Further, it should be noted that for the comparative Cₜ method, or ΔΔCₜ method, that the relative concentration of a target genomic sequence to a calibrator is:

X_{N,t}/X_{N,c}=(1+E)^{-ΔΔCt} where: (EQ.4)
X_{N,t}/X_{N,c}=is the relative concentration of the target relative to the calibrator; and
ΔΔCₜ=is the normalized difference in threshold cycles for the target and the calibrator
   Then, as previously mentioned, as assays are optimized to ensure a maximum in the reaction efficiency, or an efficiency of 1, then EQ. 4 simplifies to the calculation known to one of ordinary skill in the art for the comparative Cₜ method previously given:

X_{N,t}/X_{N,c}=2^{-ΔΔCt} (EQ. 5)

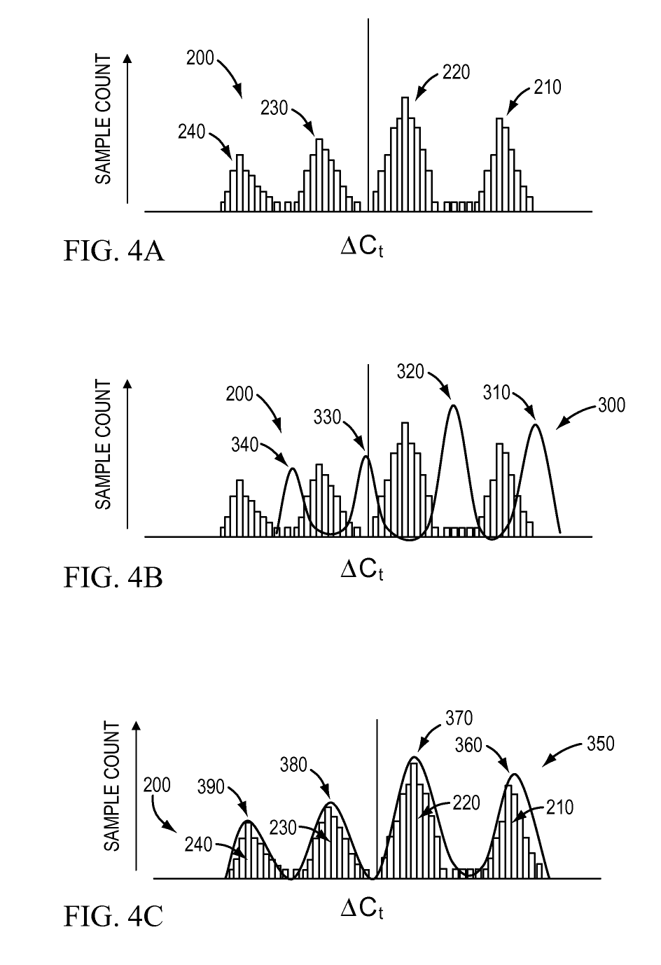

For various embodiments of methods for the determination of a copy number disclosed herein, a first step toward assigning a copy number to a sample may include the construction a frequency distribution of ΔCₜ values for a plurality of samples having different copy numbers of a target genomic, as depicted in steps 40 of FIG. 1. In that regard, in FIG. 3A, an example is shown for a frequency distribution of ΔCₜ values for a hypothetical sample population. The sample frequency distribution 200 may include distinct sample sub-distributions, such as sample sub-distributions 210, 220, 230, and 240 of FIG. 4A, where each distinct sub-distribution corresponds to a sub-distribution 210, 220, 230, and 240 that is a collection of samples having similar ΔCₜ values for a target genomic sequence. As will be discussed in more detail subsequently, such sub-distributions may be described by parameters, such as, but not limited by a sub-distribution mean and a sub-distribution variance.

According to various embodiments, as depicted in step 50 of FIG. 1, a probability density function (PDF) is selected as the basis for the assignment of a copy number of a target genomic sequence in a biological sample. As previously discussed, in various embodiments, parameters defining the selected PDF model are searched until the values for the parameters that define the PDF model are optimized to a measure of fit between the observed sample frequency distribution and the PDF model. Though a normal distribution is a probability distribution function by which many naturally occurring populations may be described, as well as being a model that is generally well -understood, any type of mono-modal distribution may be selected as appropriate for a particular observed sample frequency distribution. Accordingly, for the purpose of illustration, the normal distribution will be used in examples given subsequently. However, in addition to the normal distribution, other model monomodal distributions useful as candidates may include, but are not limited by, the Burr, Cauchy, Laplace, and logistic distributions.

According to various embodiments, an equation for copy number as a function of ΔCₜ data generated from qPCR assays having a monomodal PDF sub-distribution for each copy number cn with mean, μ_{ΔCt}(cn), is constrained to be described as:

)μ_{ΔCt}(cn)=K−log_{(1+E)}(cn) where: (EQ. 6)
μ_{ΔCt}(cn)=is the mean of the ΔCₜ sub-distributions as a function of copy number; where cn is a non-zero positive integer
K=is a constant; and
log_{(1+E)}(cn)=the log to the base (1+E) of copy number cn where E is the efficiency of the PCR amplification of the gene of interest
   as a result of EQ. 2, where, as previously described, variation in ΔCₜ data around μ_{ΔCt}(cn) may arise within and between samples with the same copy number due to various factors such as, for example, but not limited by, thermal fluctuations in the thermal cycler, and binding behaviors of PCR primers and probes. In various embodiments, an exemplary PDF model may be a normal distribution and, in this case, the full PDF model can be directly characterized by μ_{ΔCt}(cn), K, E, the sample variance, σ, and the probability of each copy number. Though these parameters directly characterize a PDF using the exemplary normal distribution, it should be understood, as previously mentioned, that any mono-modal distribution PDF may be used so long as the mean of the PDF is constrained to follow EQ.6. Accordingly, it should be understood that various mono-modal PDFs, such as, but not limited by, the normal, the Burr, Cauchy, Laplace, and logistic distributions may have different sets of parameters that characterize such model PDF distributions.

According to various embodiments, as depicted in step 60 of FIG. 1, each sample having a ΔCₜ for a target genomic sequence used to construct a frequency distribution may be assigned a copy number for that target genomic sequence based on optimizing a measure of fit between the probability density function model and the sample frequency distribution, as depicted in FIG. 4B and FIG. 4C. In FIG. 4B, model distribution 300, including sub-distributions 310, 320, 330, and 340, represents one of a plurality of distributions constructed by varying parameters characterizing a PDF model as selected in 50 of FIG. 1. In FIG. 4C, model distribution 350, including sub-distributions 360, 370, 380, and 390, represents still another of a plurality of distributions constructed by varying parameters characterizing the selected PDF model.

In various embodiments, a parameter space of parameters defining a PDF model as selected in 50 of FIG. 1 may be searched to generate a plurality of model distributions such as model distributions 300 and 350 of FIG. 4B and FIG. 4C, respectively. In various embodiments, a metric of merit may be selected to optimize the measure of fit between a PDF model distribution, such as model distributions 300 and 350 of FIG. 4B and FIG. 4C, respectively. Various embodiments may have sub-distributions described, for example, by parameters μ_{ΔCt}(cn), K, E, σ, and copy number probabilities Π_{cn}, which directly characterize a PDF based on normal sub-distributions. According to various embodiments, the parameter space may be searched using techniques, such as, but not limited by, grid searching, gradient searching, conjugate gradient searching, simulated annealing searching, genetic algorithms searching, and non-linear least squares searching. In various embodiments, the search may be initialized with a prescribed number of copy numbers associated with non-zero probability that may or may not be revised during the search. In various embodiments, the probabilities for each copy number may be initialized with all non-zero probabilities set to the same value or any other prescribed distribution and these probabilities may be revised during the search. In various embodiments, the metric of merit selected to evaluate the fit of the model PDF to the sample frequency distribution may be derived from, for example, but not limited by, the entropy (information theoretic) figure of fit, the squared differences figure of fit, and probability density figure of fit. In various embodiments using the squared difference figure of fit, the metric of merit for evaluating fit is based on a minimum value of the squared differences between the sample sub-distributions and the model PDF. For various embodiments using the entropy or probability density metrics are based on maximizing the sum of these measures across the samples evaluated with the candidate model PDF. Once the parameters that define the model PDF are determined through this process, copy numbers may be assigned to the sample sub-distributions, and hence to the samples comprising the sub-distributions.

According to various embodiments, for a calculated model based on a PDF model as selected in 50 of FIG. 1, for example as depicted in 300 FIG. 4B, and defined by a first set of parameters K₁, E₁, and Π_{cn,1} the assumption may be made that σ₁ is identical for all sub-distributions 310, 320, 330, and 340 in calculated model 300. Similarly, for any subsequent calculated model, for example as depicted in 350 FIG. 4C, and defined by a second set of parameters K₂, E₂, σ₂, and Π_{cn,2} then σ₂ may be identical for all sub-distributions 360, 370, 380, and 390 in calculated model 350. In various embodiments, the parameter space of parameters, for example such as K, E, σ, and Π_{cn} for a PDF model defined by EQ. 5, may be searched using a grid search. For various embodiments, a metric of merit, such as maximizing the probability of the observed sample frequency distribution against the PDF model distribution, may be used to asses the fit between population 200 and the calculated models 300 and 350 of FIG. 4B and FIG. 4C, respectively. For the calculated model 300 of FIG. 4B, it is apparent in this representation that the fit of the sample population 200 to the calculated model 300, constructed using a selection of a first set of parameters K₁, E₁,σ₁ and Π_{cn,1} is not good. For various embodiments, when the process is complete, a fit is then indicated by the metric of merit selected. For example, the fit of the sample population 200 to the calculated model 350 is a good fit. In such a case, the parameters of the best fit model are parameters that also estimate the sample frequency distribution. For the example shown in FIG. 4C, the second set of parameters K₂, E₂, σ₂, and Π_{cn,2} used to calculate model 350 are also parameters that estimate the sample frequency distribution. According to various embodiments, such parameters may be used to assign a copy number to sub-distributions such as sample sub-distributions 210, 220, 230, and 240 of sample distribution 200 depicted in FIGS. 4A-4C.

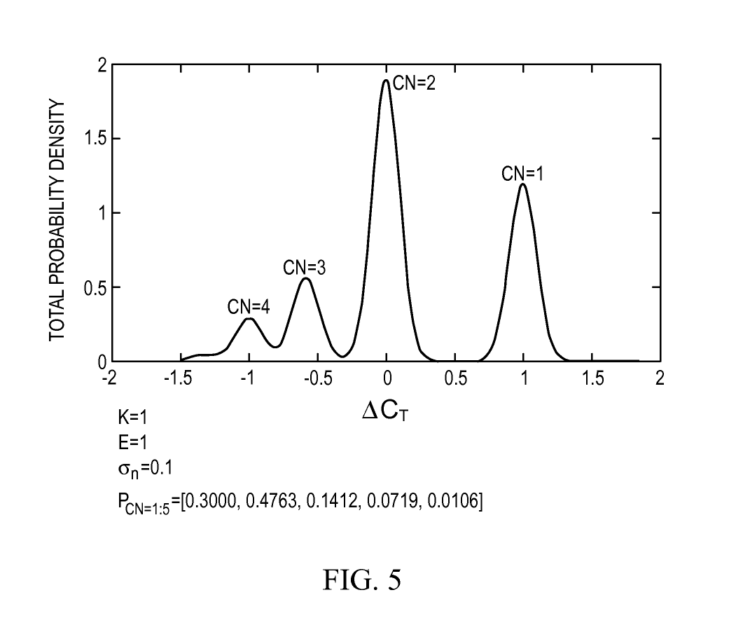

FIG. 5 shows an exemplary determination of copy number for a set of samples using various embodiments of statistical method. The PDF shown FIG. 5 was estimated from a set of 32 plates that contained data from 250 individuals and four different genes from the C4 region of the genome. As is apparent from inspection of FIG. 5, the PDF for this sample set covers the range from copy numbers (CNs) 1 to 5. The assumption of normality for the individual sub-distributions was tested by examining the sub-distribution of the samples for the sub-distribution at CN=2, which represented a set of 542 assays. To test the assumption of normality, Lilliefors test was used, with the results that 71.8% of the members of samples for the sub-distribution at CN=2 pass the test. The test of normality may not meaningful for higher CN values in the exemplary PDF of FIG. 5 because of insufficient data. A characteristic of the exemplary distribution of FIG. 5, which is exemplary of various methods utilizing the fit to a PDF, is that the separation between the μCN's decrease as CN increases. This is a direct consequence of the logarithmic relationship between ΔCₜ and the concentration of genomic material within the context of PCR. As a result of the decreasing separation between sub-distributions with increasing CN, the variability of ΔCₜ values has a larger impact on the resolution of the higher CN values. As measurement variability increases, the average confidence of higher CN calls will decrease much faster than confidence values for lower CN's. Additionally, as will be discussed in more detail subsequently, the relative probability of a copy number, the P_{CN}, can influence the confidence value associated with a call. An approximate trend is that the confidence of copy calls increases with increases in the frequency of samples belonging to that CN group. Various embodiments may be used to specify optimum ΔCₜ decision boundaries for CN value assignment. As is depicted in FIG. 5, it is apparent that these boundaries should be placed at the minimum PDF values between the peaks of the PDF since, to either side of these boundaries there is a larger likelihood that the CN corresponds to that of the closer peak in the PDF.

As depicted In FIG. 1, step 70, after the set of sample sub-distribution populations included in the sample frequency distribution have copy numbers assigned, thereby assigning copy numbers to every sample included in each sample sub-distribution, a confidence value for every sample in the sample frequency distribution may be determined.

According to various embodiments, the confidence that the assigned copy number is the true copy number within the assumption that the PDF model is accurate may be described most generally by the probability that this is so as described in the following equation:

$$\begin{matrix}P ( {cn}_{assigned} = {cn}_{true} ) = P ( {cn}_{assigned} \vert Δ {C}_{r} ' s ) \\ = P ( Δ {Ct}_{r} ' s \vert {cn}_{assigned} ) P ( {cn}_{assigned} ) \text{/} ( Δ {Ct}_{r} ' s ) \\ = \frac{{\prod }_{{cn}_{assigned}} F ( Δ {Ct}_{r} ' s \text{;} {cn}_{assigned} )}{{\sum }_{cn} {\prod }_{cn} F ( Δ {Ct}_{r} ' s \text{;} cn )} where\end{matrix} \qquad ( EQ . 6 )$$

ΔCtᵣ's refers to the replicate observations for a given person, and
F is the probability distribution function chosen for the sub-distributions that is constrained by requiring that its mean is given by:

μ_{cn}=K−log_{(1+E)}(cn)
Π_{cn} is the probability of copy number cn

As exemplary, for various embodiments where F is assumed to be a normal distribution, analyses taken from mathematical statistics can be used to produce the following:

$$P ( {cn}_{assigned} = {cn}_{true} ) = {[ 1 + {\sum }_{cn \neq {cn}_{a}} \frac{{Π}_{cn}}{{Π}_{{cn}_{a}}} {e}^{- Ω} ]}^{- 1} where subscript a is shorthand for assigned \\ {Π}_{cn} is the probability of copy number cn \\ {Ω}_{.} {≡}_{.} \frac{1}{{σ}^{2}} {log}_{( 1 + E )} ( \frac{cn}{{cn}_{a}} ) ( ( {\hat{μ}}_{r} - K ) + \frac{{log}_{( 1 + E )} ( {cn}_{a} cn )}{2} ) \\ {\hat{μ}}_{r} = \frac{1}{{N}_{r}} {\sum }_{{ all replicates}_{for a person}} Δ {Ct}_{r} ; and \\ {σ}^{2} = \begin{matrix}the variance of the sub \text{-} distributions \\ for each copy number .\end{matrix} \qquad ( EQ . 8 )$$

According to various embodiments, a confidence value may be determined by first identifying the two sample sub-distributions having the greatest number of samples, and determine the sub-distribution means for the two populations. Such a mean would be the mean of replicate means, or the mean of \hat{μ}ᵣ given above in EQ. 7. Recalling EQ. 6:

μ_{ΔCt}(cn)=K−log_{(1+E)}(cn) where: (EQ. 6)
μ_{ΔCt}(cn)=is the mean of a of the ΔCₜ sub-distributions as a function of copy number; where cn is a non-zero integer
K=is a constant; and
log_{(1+E)}(cn)=the log to the base (1+E) of the copy number of a gene in a sub-distribution of sample distributions, where E is the efficiency of the PCR amplification
   Then, for various embodiments, μ_{ΔCt}(cn) is estimated for the two populations having the greatest number of samples, yielding two independent equations, which may be used to solve for the two unknowns, K and E. Additionally, the variance for the mean of sample means, σₘₛₘ may be determined, as well as Π_{cn} the probability of copy number cn. In various embodiments, a distribution of probabilities that the assigned copy number is the true copy number may be generated using the parameters K, E, σₘₛₘ, and Πₘ. According to various embodiments, a Bootstrap technique may be used to generate such a distribution. In various embodiments, once the distribution of the probability measure given by EQ. 7 using the Bootstrap technique is generated, then a confidence level may be selected for the EQ. 7 probability measure. For example, in various embodiments a confidence level assuring that there is a 95% chance that the EQ. 7 probability is equal to or higher than the value determined for this quantity. As will be discussed in more detail subsequently, variables such as the number of samples comprising a sub-population, the copy number, and sample variance may all impact the degree to which high values for the EQ. 7 probability can be achieved.

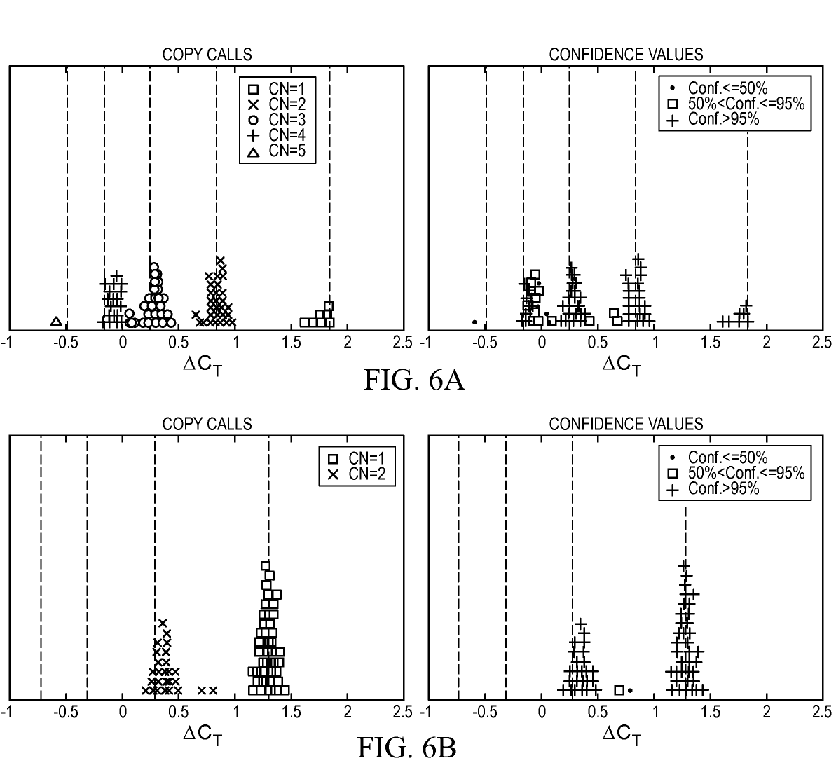

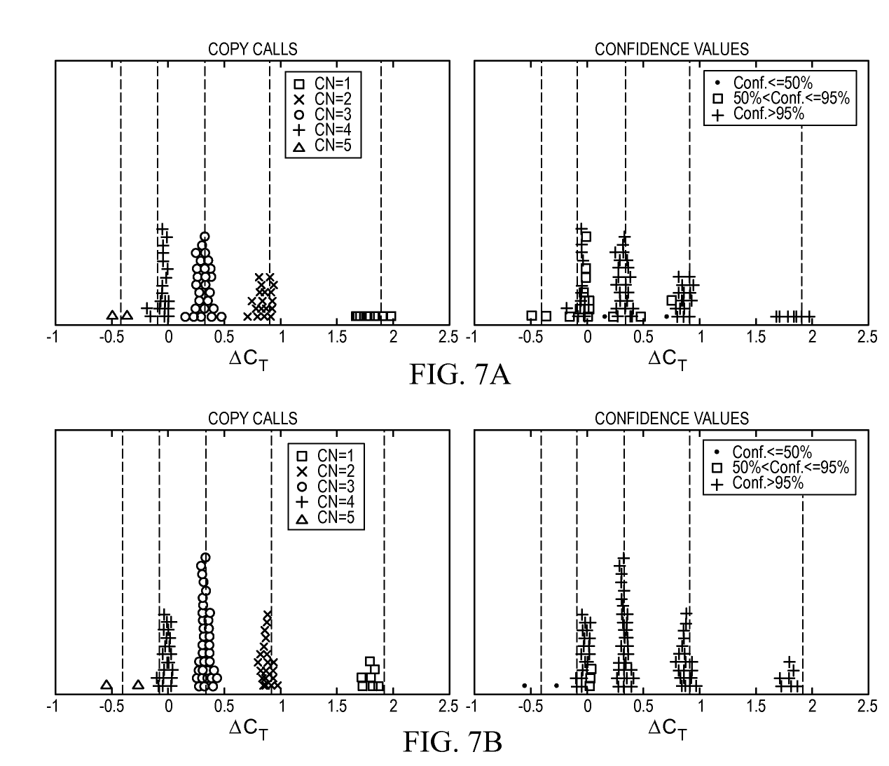

The set of figures represented by FIGS. 6A and 6B; and FIGS. 7A and 7B demonstrate the various embodiments of methods for the determination of a copy number for a genomic sequence disclosed herein. The frequency distributions represented in FIGS. 6A and 6B; and FIGS. 7A and 7B are results from 93 samples run through four different assays that target the C4 regions of the genome. For each sample, the mean ΔCₜ value across replicates is shown using a resolution of 0.05 ΔCₜ units. The height assigned to a sample has no particular significance for the sample but, over the population, the final height of points within a 0.05 ΔCₜ interval is an approximation of the relative frequency of samples that fall within that interval. The blue vertical lines show the positions of μ_{CN} according to the statistical model. For each data set by FIGS. 6A and 6B; and FIGS. 7A and 7B, there are two panels. In the left panel, the CN assignments are shown, while in the right panel, the confidence values are represented. The standard deviations shown above the plots are estimated from the data shown.

According to various embodiments, both copy number and sample variance may have an impact on the determination of a copy number, which is evident from the inspection of FIGS. 6A and 6B; and FIGS. 7A and 7B. The populations represented in FIGS. 6A and 6B have similar variance; where the variance for FIG. 6A is 0.086, and the variance for FIG. 6B is 0.090, but their CN distributions differ. The sample frequency distribution represented in FIG. 6B has fewer high CN samples and more points are called with high confidence. For the comparison of the sample frequency distributions represented by FIGS. 7A and 7B, while both sample frequency distributions have similar CN distributions but variances differ. The sample frequency distribution of FIG. 7A has a sample variance (0.071) that is higher than the sample variance of sample frequency distribution of FIG. 7B (0.062). As the sample frequency distribution represented by FIG. 7B has the lower variance, it additionally has more points called with high confidence.

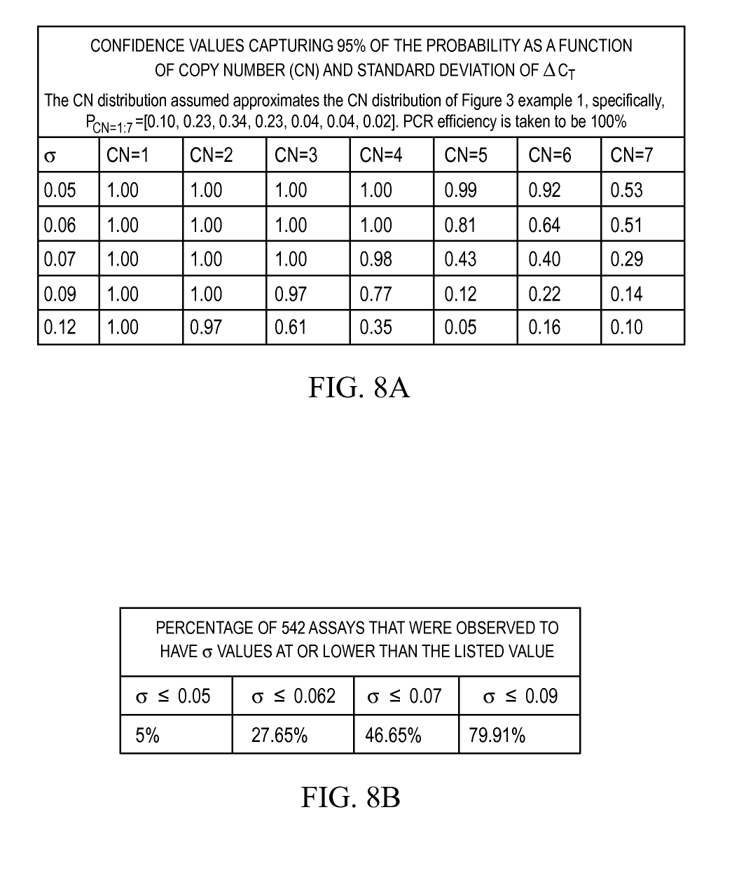

Further illustrate the impact of copy number and variance on the determination of copy number using various embodiments is illustrated in the tables presented in FIG. 8A and FIG. 8B. The illustrated in FIGS. 6A and 6B; and FIGS. 7A and 7B demonstrate that copy numbers up to 4 can be detected with high confidence if experimental conditions can be controlled to achieve a σ=0.062, such as shown in FIG. 7B. Additionally, as illustrated in the results presented in FIG. 7A, up to 3 copy numbers can be detected with a σ=0.071. In support of such results, the theoretical results presented in FIG. 7A based on various embodiments of the statistical model appear to be consistent with these observations. FIG. 8A illustrates the confidence value that can be expected for 95% or more of the samples as a function of copy number (CN) and σ, according to various embodiments. The copy number distribution used in the computations is similar to the distribution of the experimental data shown in FIG. 7B. For the theoretical results presented in FIG. 8A, for σ=0.06, the computations suggest that up to four copies can be detected with a high level of confidence. At σ=0.05, the theoretical results suggest that it is possible to detect 6-copy samples with a high level of confidence. FIG. 8B provides some empirical evidence that for various embodiments of a statistical model for the determination of a copy number, it is practical to achieve these resolution levels: In FIG. 8B, the percentage of assays achieving the listed values for σ is presented. Combining the data shown in FIGS. 8A and 8B, it is clear that for various embodiments of a statistical model for the determination of a copy number, most assays should be able to resolve CN=3, many can resolve CN=4, and it is possible to resolve CN=6.

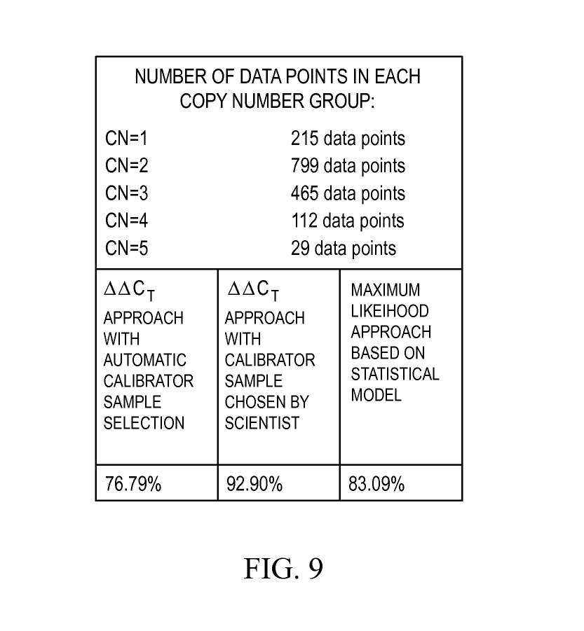

In FIG. 9, the comparison of various embodiments of a statistical model for the determination of a copy number to the ΔΔCₜ approach is shown. The data used for the comparison was generated using a set of 200 assays with 7 to 37 aneuploidy samples per plate; hence, the copy number value for each sample was known. These data were analyzed in three ways. The first analysis was the ΔΔCₜ approach using the median ΔCₜ value over the data set as the calibrator value with the assumption that it corresponds to two copies of the target gene. The second analysis was the ΔΔCₜ approach using a sample assigned by a scientist as the calibrator; the CN for this sample was also assigned by the scientist. The third analysis assigned CN values by an embodiment of a statistical model for the determination of a copy number, wherein the embodiment requires pre-specification of the CN value that is expected to occur most frequently. FIG. 9 shows the results of these analyses.

In addition to the steps of various embodiments shown in FIG. 1, one of ordinary skill in the art would recognize additional steps that may be routinely performed on qPCR data, for example, but not limited by, the steps of validating and filtering the data, as well as excluding outliers. In various embodiments, detecting outliers from replicates is performed by assuming that the variation of replicates about the replicate mean is the same for all samples, estimating this variation using data from all samples excluding replicates that deviate from the replicate median by more than a selected amount, and labeling a replicate as an outlier if it differs from the replicate median by more than a selected number of standard deviations. According to various embodiments, the selection may be done based on the square root of the variance. As demonstrated above in FIGS. 6A and 6B; FIGS. 7A and 7B, and FIGS. 8A and 8B, the sample variance may have a significant impact on results for determining the copy number of target genomic sequence; either a gene or genomic sequence of interest. In various embodiments, the exclusion of verified outliers may then provide additional robustness to the copy number determination.

While the principles of this invention have been described in connection with specific embodiments, it should be understood clearly that these descriptions are made only by way of example and are not intended to limit the scope of the invention. What has been disclosed herein has been provided for the purposes of illustration and description. It is not intended to be exhaustive or to limit what is disclosed to the precise forms described. Many modifications and variations will be apparent to the practitioner skilled in the art. What is disclosed was chosen and described in order to best explain the principles and practical application of the disclosed embodiments of the art described, thereby enabling others skilled in the art to understand the various embodiments and various modifications that are suited to the particular use contemplated. It is intended that the scope of what is disclosed be defined by the following claims and their equivalence.

What is claimed is:

1. A method for determining the copy number of a genomic sequence in a biological sample, the method comprising:
calculating a ΔCₜ value for a target genomic sequence for each sample in a set of biological samples using an endogenous reference genomic sequence;
constructing a frequency distribution of the ΔCₜ values calculated for each sample, wherein the frequency distribution comprises a set of sub-distributions of ΔCₜ values for the target genomic sequence in a set of biological samples; and
determining a copy number for the target genomic sequence for each sample in the set of biological, wherein the determination is an assignment of a copy number for each sample based on a measure of fit between a probability density function model and the frequency distribution.

2. The method of claim 1, wherein the method further comprises calculating a confidence value for the copy number determined for the first target genomic sequence for each sample in the set of biological samples.

3. The method of claim 2, wherein the confidence value is an estimate of the probability that the assigned copy number for the target genomic sequence of each sample is the correct copy number based on the probability density function model at a designated confidence level.

4. The method of claim 1, wherein the probability density function model is a monomodal distribution model.

5. The method of claim 4, wherein the probability density function model is a normal distribution model.

6. The method of claim 4, wherein the probability density function model is selected from Burr, Cauchy, Laplace, and logistic distribution models.

7. The method of claim 1, wherein the method further comprises filtering the data to exclude outliers.

8. A computer implemented method of determining a copy number of a biological sample, the method comprising:
obtaining ΔCₜ data for a target genomic sequence for each sample in a set of biological samples;
processing the ΔCₜ data on a computer to determine a copy number for each sample in a biological, the processing comprising:
constructing a frequency distribution of the ΔCₜ values, wherein the frequency distribution comprises a set of sub-distributions of ΔCₜ values for the target genomic sequence in a set of biological samples;
determining a copy number for the target genomic sequence for each sample in the set of biological, wherein the determination is an assignment of a copy number for each sample based on a measure of fit between a probability density function model and the frequency distribution; and
outputting the assigned copy numbers to an end user.

9. The method of claim 8, wherein the method further comprises calculating a confidence value for the copy number determined for the first target genomic sequence for each sample in the set of biological samples.

10. The method of claim 9, wherein the confidence value is an estimate of the probability that the assigned copy number for the target genomic sequence of each sample is the correct copy number based on the probability density function model at a designated confidence level.

11. The method of claim 8, wherein the probability density function model is a monomodal distribution model.

12. The method of claim 11, wherein the probability density function model is a normal distribution model.

13. The method of claim 11, wherein the probability density function model is selected from Burr, Cauchy, Laplace, and logistic distribution models.

14. The method of claim 8, wherein the method further comprises filtering the data to exclude outliers.

15. A computer program product comprising:
a computer-readable medium and computer-readable code embodied on said computer-readable medium for determining a copy number of a biological sample, the computer-readable code comprising:
obtaining ΔCₜ data for a target genomic sequence for each sample in a set of biological samples;
constructing a frequency distribution of the ΔCt values, wherein the frequency distribution comprises a set of sub-distributions of ΔCt values for the target genomic sequence in a set of biological samples; and
determining a copy number for the target genomic sequence for each sample in the set of biological, wherein the determination is an assignment of a copy number for each sample based on a measure of fit between a probability density function model and the frequency distribution

16. The method of claim 15, wherein the method further comprises calculating a confidence value for the copy number determined for the first target genomic sequence for each sample in the set of biological samples.

17. The method of claim 16, wherein the confidence value is an estimate of the probability that the assigned copy number for the target genomic sequence of each sample is the correct copy number based on the probability density function model at a designated confidence level.

18. The method of claim 15, wherein the probability density function model is a monomodal distribution model.

19. The method of claim 18, wherein the probability density function model is a normal distribution model.

20. The method of claim 18, wherein the probability density function model is selected from Burr, Cauchy, Laplace, and logistic distribution models.
